(19) United States
(12) Patent Application Publication
(10) Pub. No.: US 2008/0313838 A1
(43) Pub. Date: Dec. 25, 2008

(54) Scratcher

(76) Inventor: Toru Nakatani, Gero-shi (JP)

Correspondence Address: FLYNN THIEL BOUTELL & TANIS, P.C., 2026 RAMBLING ROAD, KALAMAZOO, MI, 49008-1631, US

(21) Appl. No.: 11/985,223

(22) Filed: Nov. 14, 2007

(30) Foreign Application Priority Data
Jun. 19, 2007 (JP) JP 2007-160925

(51) Int. Cl. B08B 1/00; A61F 11/00; B08B 9/00

(52) U.S. Cl. 15/236.01; 606/162

(57) ABSTRACT

To definitely remove stains in a small and narrow clearance. Thread 2 is spirally wound around a wire 1 formed by material with plasticity, the two or more thread-wound wires obtained are twisted together in spirals to bring the thread 2 in close contact with the wire 1 and the overall diameter is held to be small. In addition, with the winding thread, spiral narrow-width protrusions and recesses are formed, with the thread-wound wire, spiral wide-width protrusions and recesses are formed, and by crossing these and allowing a large number of small and large protrusions and recesses to be mixed, chances to catch removed matters are increased, and a satisfactory scratching condition is achieved.

### BACKGROUND OF THE INVENTION

1. Field of the Invention

The present invention relates to a scratcher used for cleaning void parts between components of devices or narrow and small clearances such as pierced holes formed on a human body, external ear canals, etc., or a scratcher useful for pathologic practices or microbiological treatment, for example, sampling cells of patients' lesions, inoculation of strains at the time for culture processing of bacteria.

2. Description of the Related Art

In the past, as cleaning tool of pierced holes, an invention that has an insertion member formed by integrally twisting multiple wires and a handle section with this insertion member provided in a protruding condition on its one end (see the following patent document 1) has been observed.

This cleaning tool has voids between wires which form spirals, and specifically, an invention which has an insertion member with spiral concaves and convexes alternately formed on the surface by integrally twisting multiple metal wires whose cross-sectional surface is nearly circular and a handle section with this insertion member protrudably installed on its one end was presented.

These inventions retain antiseptic solutions in the concaves as voids between wires and provide outstanding detergent properties and at the same time, they can efficiently scrape out ear wax, etc. by the convexes.

Furthermore, an invention which has an insertion member with a brush section that has a flexible body spirally protruded around the spiral axis and a handle section with this insertion member protrudably installed on its one end by integrally twisting multiple metal wires whose cross-sectional surface is nearly circular with a wire-form or plate-form flexible body placed in-between was presented, too, in order to enhance the detergent properties and scrape-out efficiency.

[Patent document 1]

Japanese Patent Application Laid-Open 2004-49596 (claims and FIG. 1 through FIG. 5)

However, the cleaning tool which has the brush section is unsuited for being inserted into a clearance narrower and smaller than a pierced hole because the flexible body protrudes in its radial direction, and when it is used, the flexible body bends and the catch between the flexible body and the removed substance (ear wax, etc.) is disengaged, and the removed substance is left behind.

### SUMMARY OF THE INVENTION

In view of the problem above, the present invention brings the thread in close contact with the wire and keeps the overall diameter small by forming the present invention by spirally winding a thread around the wire composed of material with plasticity and spirally twisting two or more thread-wound wires obtained.

In addition, spiral-form narrow-width recessed and protruded portions are formed by the winding thread, spiral-form wide-width recessed and protruded portions are formed by thread-wound wires, and these are crossed and small and large recessed and protruded portions are mixed in a large quantity; thereby catches of removed material are increased and the scraping conditions are made satisfactory to solve the above-mentioned problem.

In short, because the present invention is formed by spirally winding thread around a wire composed by material with plasticity and spirally twisting two or more thread-wound wires obtained, the thread comes in close contact with the wire, and the overall diameter can be reduced, and the present invention can be inserted into an extremely narrow and small clearance.

Furthermore, liquids such as medical solutions for cleaning can be immersed and retained in the wound thread, and moreover, the liquid formulation can be retained by the spiral narrow-width recessed and protruded portions formed by winding thread and the spiral wide-width recessed and protruded portions formed by the thread-wound wires, too, and a large quantity of liquid formulation can be retained and cleaning between narrow and small clearances can be facilitated.

Furthermore, by the narrow-width recessed and protruded portions, small removed substances are caught, while by the wide-width recessed and protruded portions, large removed substances can be caught, and furthermore, since the narrow-width recessed and protruded portions and wide-width recessed and protruded portions are crisscross arranged, the removed substance can be efficiently scratched off by these combinations regardless of how the scratcher is moved.

Because one thread-wound wire is folded into two and the two segment portions are twisted from the turning point to both ends, easy twisting (manufacture) can be achieved, and by bringing the turning point to the head end side, the head end side is not pointed but smoothened, securing safety when the scratcher is used for cleaning a human body portion such as pierced holes, external ear canals, etc.

Because the thread has protrusions on the circumferential surface and angular portions are formed on the protruded end face edge of the protrusions, removed substances are definitely caught by the angular portions and the wipe-off capabilities can be enhanced.

Because an annular portion is protrudably provided on one end of the scratcher, by further bringing the annular portion to the head safety is enhanced in such cases when the scratcher is used for cleaning human body portions such as pierced holes, external ear canals, and can catch and remove even large removed substances which are unable to be caught with the wide-width recessed and protruded portions.

In addition, in the event that the scratcher is used for pathological practice and microbiological treatment, still larger or still greater amount of substances can be caught not only by the spiral recessed and protruded portions but also by the annular portion.

Because the whole scratcher is rolled to be flat, rigidity can be secured by flattening in the width direction of the flattened body and at the same time, the thickness can be reduced or slenderized in the thickness direction from the wire diameter before rolling to be flat, and therefore, for example, in the event that the scratcher is used for a slit-like clearance, the scratcher can conform to a still narrower and smaller clearance.

Because a liquid absorber such as swab, etc. is attached to the other end of the scratcher, for example, after cleaning the external ear canal by the liquid absorber such as cotton-tipped swabs after taking bath and removing moisture together with ear wax, dry fine ear wax remaining in the external ear canal can be removed further with the opposite side. In this way, great practical effects can be achieved, because applications of the scratcher can be increased such as applying it to cleaning of wet portions as described above.

### BRIEF DESCRIPTION OF THE DRAWINGS

FIG. 1 is a schematic front view of a thread-wound wire according to the present invention;

FIG. 2 is a schematic front view that shows a condition in which the thread-wound wire of FIG. 1 is folded into two;

FIG. 3 is a schematic front view of a scratcher with an annular portion provided on one end thereof;

FIG. 4 is a partially enlarged view of a thread-wound wire;

FIG. 5 is a cross-sectional view of thread cut in the radial direction;

FIG. 6 is a cross-sectional view of thread cut in the length direction; and

FIG. 7 is a schematic front view of a scratcher with an absorber provided on the other end.

### DESCRIPTION OF THE PREFERRED EMBODIMENTS

Now, embodiments are shown as follows for the scratcher according to the present invention, and the scratcher is described in detail.

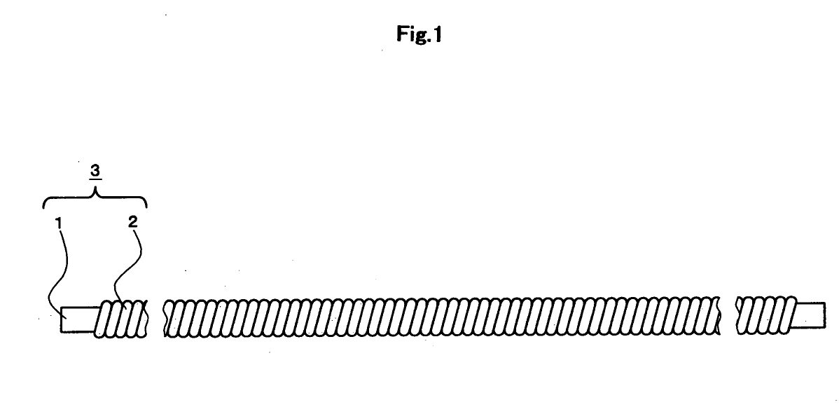

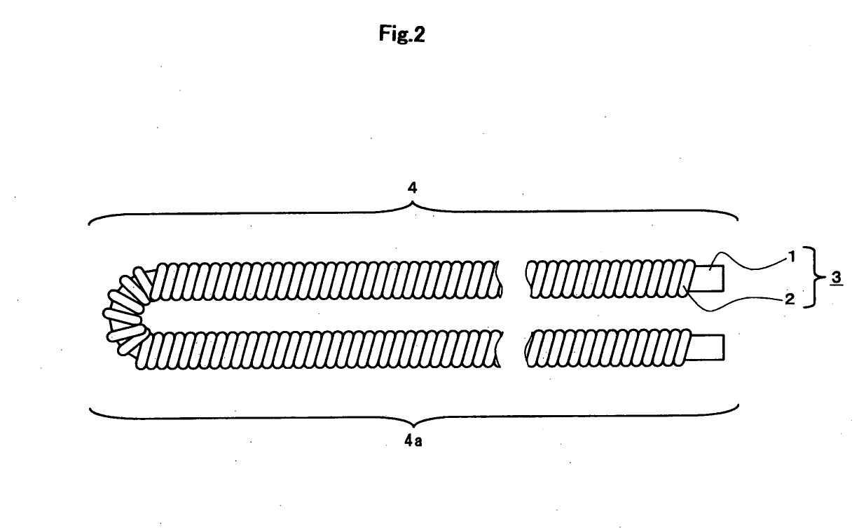

The scratcher of the present embodiment is formed by spirally winding small-diameter thread 2 around a wire 1 as shown in FIG. 1, folding the obtained thread-wound wire 3 into two as shown in FIG. 2, and spirally twisting the two segment portions 4 and 4a from the turning point to both ends.

On the turning point side, an annular portion 5 is formed by the portion of the two segment portions 4 and 4a left untwisted.

Because the thread 2 is fixed by being pinched between wires 1, no adhesive is required, but the thread 2 may be wound after applying adhesives to the wire 1 so that they are brought into stronger contact and are fixed.

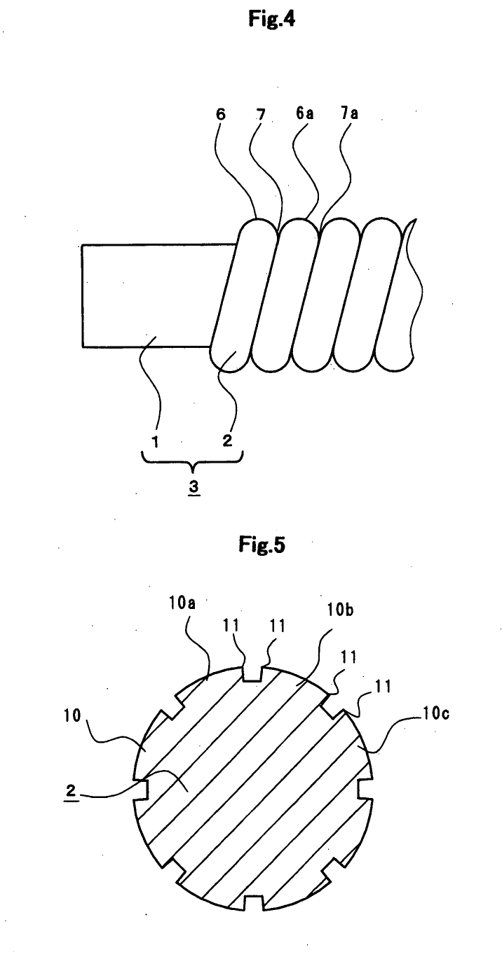

As shown in FIG. 4, this scratcher has small protrusions 6, 6a, . . . on the circumferential surface of the wire 1 by thread 2, small recesses 7, 7a, . . . between the small protrusions 6, 6a, . . . appear alternately on the circumferential surface of the wire 1, and narrow-width recesses and protrusions are formed by spirally winding the thread 2 around the wire 1.

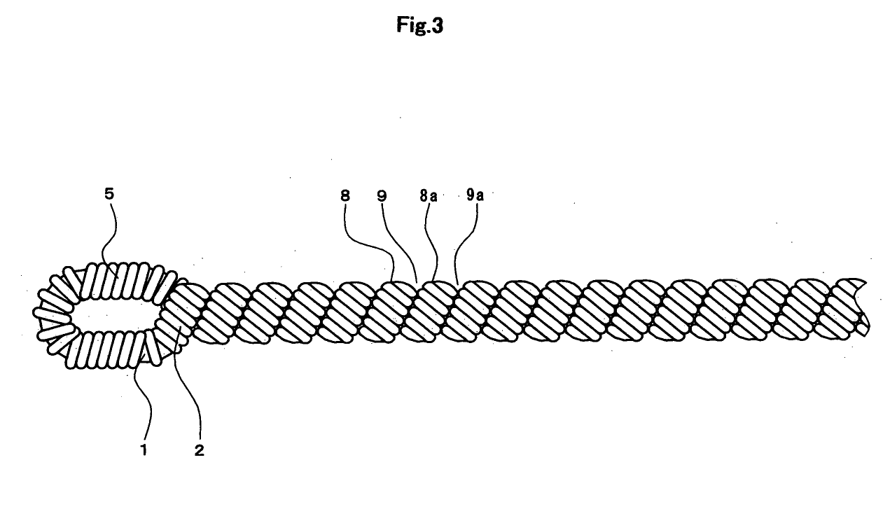

In addition, as shown in FIG. 3, by spirally twisting the thread-wound wires 3, large protrusions 8, 8a, . . . by the thread-wound wires 3 and large recesses 9, 9a, . . . between the large protrusions 8, 8a, . . . appear alternately and large-width recesses and protrusions are formed, and these wide-width recesses and protrusions and the narrow-width recesses and protrusions are crossed, and a large number of small and large recesses and protrusions exist on the circumferential surface of the scratcher, and by these small and large recesses and protrusions as well as the above-mentioned annular section 5, removed substances are caught.

The wire 1 is formed by material with plasticity which is able to be twisted together, for example, metal, synthetic resin, and composite material of them, etc., and allowing the scratcher to memorize the shape of the scratcher after twisting by the use of material with shape-memory functions can restore the shape by heating, etc. even when the scratcher has been bent, and the scratcher can be repeatedly used by washing.

A thread 2 may be a strand made of natural fibers (cotton, linen, silk, etc.), semi-synthetic fibers (rayon, etc.), synthetic fibers (polyamide, polyvinyl alcohol, polyester, polyolefin, polyurethane, polyamide, etc.), inorganic fibers (glass fiber, carbon fiber, boron fiber, etc.), and others as well as a single yarn of them, and it does not matter whether or not the thread 2 provides liquid absorbing capabilities.

By the way, in order to secure a greater retention amount of a liquid formulation, for example, a thread 2 of special synthetic fibers with particularly high water absorbing capabilities, such as LANSEAL (registered trademark) commercially available from Toyobo Co., Ltd., AQUSIA available from KB Seiren, Ltd., BELL OASIS (registered trademark) available from Teijin Fibers Ltd., and others may be used, or, alternatively, highly liquid absorbing resins and compounds (for example, polyacrylic acid, polyacrylate, sodium hyaluronate, cross-linked polyglutamic acid, etc.) may be immersed and solidified to thread 2 with liquid absorbing capabilities, or may be applied to the surface of the thread 2 of non-liquid absorbing capabilities, or may be used as adhesives used for anchoring the thread 2 to a wire 1.

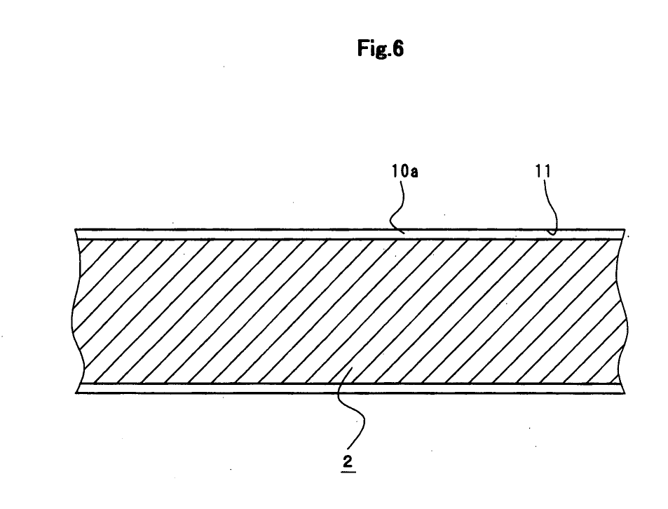

Furthermore, it is preferable that a thread 2 has protrusions on its peripheral surface and have angular portions formed at the protruded end face edges of the protrusions, and more specifically, for example, as shown in FIG. 5 (cross-sectional view in the thread radial direction) and FIG. 6 (cross-sectional view in the thread length direction), multiple convexes (protrusions) 10, 10a, . . . may be formed along the thread length direction and angular portions 11 may be formed at the edge of the top end faces of the relevant convexes 10, 10a, or though not illustrated, convexes may be formed along the thread circumference at appropriate intervals in the thread length direction, and by using these kinds of thread 2, removed substances can be definitely caught at the angular portions 11.

By the way, for an example of the thread of the shapes shown in FIG. 4 and FIG. 5, Belima (registered trademark) X commercially available from KB Seiren, Ltd. can be mentioned.

As described above, in the present embodiment, the scratcher is formed with one thread-wound wire 3 but may be formed with two independent thread-wound wires 3, or by using three or more wires, the rigidity may be secured, though the scratcher becomes slightly thick, or the entirety of such scratchers may be rolled and flattened to conform to a narrow and small slit-like clearance, and the diameter and the length of the scratchers may be set appropriately in accordance with the intended uses.

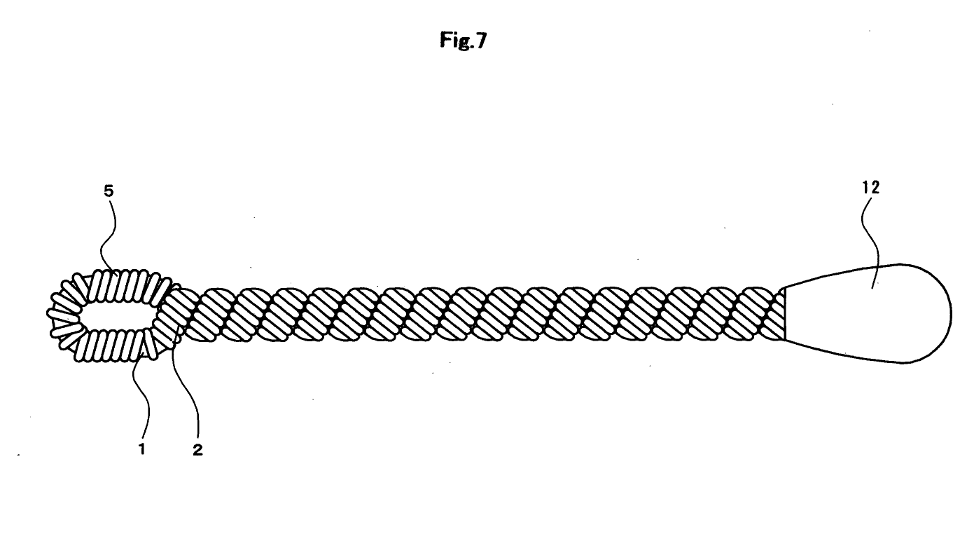

In addition to the above configurations, a liquid absorber 12 may be provided on one end of the scratcher, and in the case of a scratcher with an annular part 5 provided on one end as shown in FIG. 7, the liquid absorber 12 may be provided on the other end.

For the liquid absorber 12, swabs used for cotton-tipped swabs, sponges made of synthetic resins, etc. or unwoven fabric may be wound to be used for the liquid absorber 12.

1. A scratcher, comprising:
thread which is spirally wound around a wire comprising material with plasticity; and
two or more thread-wound wires which are spirally twisted.

2. The scratcher according to claim 1, wherein one thread-wound wire is folded into two and the two segment portions are twisted from the turning point to both ends.

3. The scratcher according to claim 1, wherein the thread has protrusions around the circumferential surface and angular portions are formed at the protruded end face edge of the protrusions.

4. A scratcher characterized by protrudably installing an annular portion on one end of the scratcher according to claim 1.

5. A scratcher characterized by rolling to flatten the scratcher according to claim 1.

6. A scratcher characterized by installing a swab and other liquid absorbers on the other end of the scratcher according to claim 1.
